(19) United States
(12) Patent Application Publication
(10) Pub. No.: US 2010/0216683 A1
(43) Pub. Date: Aug. 26, 2010

(54) Cleaning Product

(76) Inventors: Arnd Kessler, Monheim (DE); Nina Viola Reichhardt, Lund (SE); Nadine Warkotsch, Dusseldorf (DE); Johannes Zipfel, Dusseldorf (DE); Christian Nitsch, Dusseldorf (DE)

Correspondence Address: Henkel Corporation, 10 Finderne Avenue, Bridgewater, NJ, 08807, US

(21) Appl. No.: 12/707,684

(22) Filed: Feb. 18, 2010

Related U.S. Application Data
(63) Continuation of application No. PCT/EP2008/056344, filed on May 23, 2008.

(30) Foreign Application Priority Data
Aug. 22, 2007 (DE) 10 2007 039 655.6

(51) Int. Cl. C11D 1/72; C12N 9/96

(52) U.S. Cl. 510/221; 510/422; 510/393; 435/188

(57) ABSTRACT

Liquid, aqueous washing or cleaning product formulation, comprising a) at least one washing- or cleaning-active enzyme; b) 1,2-propylene glycol; and c) nonionic surfactant of the general formula R¹—CH(OH)CH₂O-(AO)_{w}-(A′O)ₓ-(A′O)_{y}-(A″O)_{z}—R², wherein R¹ is a straight-chain or branched, saturated or mono- or polyunsaturated C₆₋₂₄-alkyl or -alkenyl radical; R² is a linear or branched hydrocarbon radical having from 2 to 26 carbon atoms; A, A′, A′ and A″ are each independently a radical from the group of —CH₂CH₂, —CH₂CH₂—CH₂, —CH₂—CH(CH₃), —CH₂—CH₂—CH₂—CH₂, —CH₂—CH(CH₃)—CH₂—, and —CH₂—CH(CH₂—CH₃); and w, x, y and z are each values from 0.5 to 120, where x, y and/or z may also be 0, are notable for good phase and enzyme stability and good cleaning performance.

### CROSS REFERENCE TO RELATED APPLICATIONS

The present application is a continuation of International Application No. PCT/EP2008/056344 filed 23 May 2008, which claims priority to German Patent Application No. 10 2007 039 655.6 filed 22 Aug. 2007, both of which are incorporated herein by reference.

The present application describes washing and cleaning agents, in particular, enzyme-containing washing and cleaning agents.

Manufactured and presentation forms of washing and cleaning agents are constantly subject to new modifications. This includes, for example, the convenient metering of washing and cleaning agents and simplification of the process stages required for carrying out a laundering or cleaning procedure.

In this context, devices for multi-dosing of washing and cleaning agents have recently become a focus of attention for the product developer. These devices include metering containers integrated into the automatic dishwasher or washing machine, as well as devices that are independent from the automatic dishwasher or washing machine. Over the course of a plurality of sequential cleaning stages, portions of washing or cleaning agent are automatically or semi-automatically metered into the interior of the cleaning machine by these devices containing the multiple doses of cleaning agent required for carrying out a cleaning process. Examples of such devices are described in European patent application EP 1 759 624 A2 (Reckitt Benckiser) and German patent application DE 10 2005 062 479 A1 (BSH Bosch and Siemens Hausgeräte GmbH).

Regardless of the type of metering device placed in the interior of the automatic dishwashers or washing machines, washing or cleaning agents placed in these devices for multiple metering can be exposed for a long period of time to varying temperatures, these temperatures being approximately equivalent to the water temperature used in carrying out the washing or cleaning process. These temperatures can be up to 95° C., wherein in automatic dishwashing usually temperatures from 50 to 75° C. are attained. In the course of multiple laundry or cleaning processes a washing or cleaning agent contained in a device intended for multiple dosing can be repeatedly heated to temperatures significantly above those typical for transportation and storage, with temperature-sensitive, active substances particularly affected. Such temperature-sensitive washing- or cleaning-active substances includes washing- or cleaning-active enzymes.

Use of enzymes for improving the laundry and cleaning power of washing and cleaning agents has been known for some decades. In particular, hydrolytic enzymes such as proteases, amylases or lipases, due to their direct cleaning action, can be a component of numerous cleaning agents for fabrics or dishes.

Proteases, especially serine proteases such as subtilases, serve to degrade protein-containing stains on the product being cleaned. Among the laundry washing and cleaning agent proteases, subtilisins stand out due to their favorable enzymatic properties with respect to stability or pH optimum. Within the amylase enzyme class α-amylases are prevalent. α-amylases (E. C. 3.2.1.1) hydrolyze internal α-1,4-glycosidic bonds of starch and starch-like polymers.

In washing and cleaning agents, the cleaning action of an incorporated enzyme, which is decisive for the consumer, is also determined, in addition to the enzyme structure, to a significant degree by the type of enzyme packaging and its stability against environmental influences.

Washing- or cleaning-active enzymes are found in both solid and liquid form. Solid enzyme preparations include in particular enzyme granulates consisting of a plurality of ingredients and which can be preferably incorporated into solid washing and cleaning agents. Liquid or gel type washing and cleaning agents frequently comprise liquid enzyme preparations, these being much less protected against external influences than the enzyme granulates.

Various protective measures have been proposed for increasing the stability of these enzyme-containing liquid washing or cleaning agents. Thus, for example, German patent application DE 2 038 103 (Henkel) teaches stabilization of enzyme-containing dishwasher agents by saccharides, whereas propylene glycol is disclosed in the European patent EP 646 170 B1 (Procter & Gamble) for stabilizing enzymes in liquid cleaning agents.

Methods described in the prior art for stabilizing enzymes take into account only to a limited extent the problematic nature of repeated exposure to high temperatures, such as occur in the above described devices for multiple dosing of washing or cleaning agents. These known methods are only suitable to a limited extent for avoiding loss of activity or segregation of the enzyme in liquid cleaning agents.

Accordingly, the present application provides for stabilization of a washing- or cleaning-active enzyme preparation against phase separation/loss of activity during multiple variations in temperature (10 to 75° C.). Suitable enzyme preparations should be storable without significant loss of activity in a storage device located in the interior of the automatic dishwasher or washing machine.

Surprisingly it was found that liquid enzyme preparations could be stabilized by addition of a mixture of propylene glycol and specific hydroxy mixed ether non-ionic surfactants.

Accordingly, a first subject matter of this application is a liquid, aqueous washing or cleaning agent preparation, comprising
a) at least one washing- or cleaning-active enzyme;
b) 1,2-propylene glycol; and
c) a non-ionic surfactant according to the general formula

R¹—CH(OH)CH₂O-(AO)_{w}-(AO)ₓ-(A″O)_{y}-(A′″O)_{z}—R²
wherein R¹ represents a straight chain or branched, saturated, mono- or polyunsaturated C₆₋₂₄ alkyl or alkenyl group; R² represents a linear or branched hydrocarbon group containing 2 to 26 carbon atoms; A, A′, A″ and A′″ independently represent —CH₂CH₂, —CH₂CH₂—CH₂, —CH₂—CH(CH₃), —CH₂—CH₂—CH₂—CH₂, —CH₂—CH(CH₃)—CH₂—, or —CH₂—CH(CH₂—CH₃); and w, x, y and z represent values between 0.5 and 120, wherein x, y and/or z can also be 0.

The subject matter of this application includes liquid, aqueous washing or cleaning agent preparations. “Aqueous” preparations are designated as those that comprise at least 5 wt. %, preferably at least 10 wt. % water. Preferred liquid washing or cleaning agent preparations according to the invention are those wherein the weight fraction of water is 5 to 35 wt. %, preferably 10 to 25 wt. % and in particular 12 to 30 wt. %, relative to total weight of the washing or cleaning agent preparation.

Washing or cleaning agent preparations according to the invention include at least one washing- or cleaning-active enzyme as a first ingredient. Preferred added enzymes include proteases, amylases, lipases, hemicellulases, cellulases, perhydrolases or oxidoreductases, as well as their mixtures. These enzymes are of natural origin, with improved variants based on the natural molecules being available for use in laundry agents or cleaning compositions and, as such, are preferred. The washing or cleaning compositions preferably comprise enzymes in total quantities of 1×10⁻⁶ to 5 wt. % based on active protein. Protein concentration can be determined using known methods such as the BCA Process or biuret process.

The stabilizing action according to the invention was observed in particular with amylases and proteases. Therefore, liquid washing or cleaning agent preparations according to the invention comprising an amylase and/or protease washing- or cleaning-active enzyme are preferred. Preferred proteases include those of the subtilisin type. Examples of these include the subtilisins BPN′ and Carlsberg as well as their further developed forms, protease PB92, subtilisins 147 and 309, the alkaline protease from Bacillus lentus, subtilisin DY, and subtilases enzymes no longer classified in the stricter sense as subtilisins (e.g., thermitase, proteinase K and the proteases TW3 and TW7).

Preferred liquid washing or cleaning agent preparations according to the invention comprise 5 to 50 wt. %, preferably 7 to 45 wt. % and in particular 10 to 40 wt. % protease preparations, relative to total weight of the washing or cleaning agent preparation.

Examples of further useable amylases according to the invention include α-amylases from Bacillus licheniformis, B. amyloliquefaciens, B. stearothermophilus, Aspergillus niger and A. oryzae, as well as improved developments of the cited amylases for use in washing and cleaning agents. Moreover, the α-amylase from Bacillus sp. A 7-7 (DSM 12368) and the cyclodextrin-glucanotransferase (CGTase) from B. agaradherens (DSM 9948) are also useful.

Preferred liquid washing or cleaning agent preparations according to the invention comprise 0.1 to 20 wt. %, preferably 0.2 to 15 wt. % and in particular 1.0 to 12 wt. % amylase preparations, relative to total weight of the washing or cleaning agent preparation.

Washing- or cleaning-active proteases and amylases are generally not available in their pure protein form but rather in the form of stabilized, storable and transportable preparations. These prefabricated preparations include, for example, solid preparations obtained by granulation, extrusion or lyophilization, or, enzyme solutions for liquid compositions or gel-type compositions, advantageously as highly concentrated as possible, of low moisture content and/or mixed with stabilizers or further adjuvants.

Alternatively, the enzymes can also be encapsulated, for example, by spray drying or extrusion of the enzyme solution together with a preferably natural polymer, or in the form of capsules, for example, those in which the enzyme is embedded in a solidified gel, or in those of the core-shell type, in which an enzyme-containing core is covered with a water-, air- and/or chemical-impervious protective layer. Further active principles such as stabilizers, emulsifiers, pigments, bleaches or colorants can be applied in additional layers. Such capsules are made using known methods, for example, by vibratory granulation or roll compaction or by fluidized bed processes. Advantageously, these types of granulates, for example, with an applied polymeric film former, are dust-free and, as a result of the coating, are storage stable.

In addition, it is possible to formulate two or more enzymes together so that a single granulate exhibits a plurality of enzymatic activities.

As the preceding examples demonstrate, the enzyme protein forms only a fraction of the total weight of customary enzyme preparations. Inventively preferred added protease and amylase preparations comprise between 0.1 and 40 wt. %, preferably between 0.2 and 30 wt. %, particularly preferably between 0.4 and 20 wt. % and especially between 0.8 and 10 wt. % of the enzyme protein.

According to the invention, lipases or cutinases can also be incorporated, particularly due to their triglyceride cleaving activities, but also in order to produce in situ peracids from suitable preliminary steps. These include, for example, available or further developed lipases originating from Humicola lanuginosa (Thermomyces lanuginosus), particularly those with the amino acid substitution D96L. Moreover, suitable cutinases include those originally isolated from Fusarium solani pisi and Humicola insolens. Further suitable lipases or cutinases include those whose starting enzymes were originally isolated from Pseudomonas mendocina and Fusarium solanii.

In addition, enzymes summarized under the term hemicellulases can be added. These include mannanases, xanthanlyases, pectinlyases (=pectinases), pectinesterases, pectatlyases, xyloglucanases (=xylanases), pullulanases and β-glucanases.

To increase the bleaching action, oxidoreductases such as oxidases, oxygenases, katalases, peroxidases (e.g., halo-, chloro-, bromo-, lignin-, glucose- or manganese-peroxidases, dioxygenases or laccases (phenoloxidases, polyphenoloxidases) can be incorporated according to the invention. Advantageously, additional, preferably organic, particularly preferably aromatic compounds that interact with the enzymes are added to enhance the activity of the relative oxidoreductases or to facilitate the electron flow (mediators) between the oxidizing enzymes and the stains over strongly different redox potentials.

A plurality of enzymes and/or enzyme preparations, preferably liquid protease preparations and/or amylase preparations, are preferably added.

A second ingredient of the washing or cleaning agent preparation according to the invention is 1,2-propylene glycol. The weight fraction of 1,2-propylene glycol based on total weight of the washing or cleaning agent preparations according to the invention can vary widely, although preparations have proved to be particularly stable which comprise 5 to 60 wt. %, preferably 10 to 50 wt. %, and in particular 15 to 45 wt. % 1,2-propylene glycol relative to total weight of the washing or cleaning agent preparation. Corresponding preparations are consequently preferred according to the invention.

Finally, a third ingredient of the washing or cleaning agent preparations according to the invention is non-ionic surfactants of the general formula

R¹—CH(OH)CH₂O-(AO)_{w}-(A′O)ₓ-(A″O)_{y}-(A′″O)_{z}—R²

wherein R¹ represents a straight chain or branched, saturated, mono- or polyunsaturated C₆₋₂₄ alkyl or alkenyl group; R² represents a linear or branched hydrocarbon group containing 2 to 26 carbon atoms; A, A′, A″ and A′″ independently represent —CH₂CH₂, —CH₂CH₂—CH₂, —CH₂—CH(CH₃), —CH₂—CH₂—CH₂—CH₂, —CH₂—CH(CH₃)—CH₂—, —CH₂—CH(CH₂—CH₃); and w, x, y and z represent values from 0.5 to 120, wherein x, y and/or z can also be 0.

By adding the abovementioned non-ionic surfactants of the general formula R¹—CH(OH)CH₂O-(AO)_{w}-(A′O)ₓ-(A″O)_{y}-(A′″O)_{z}—R², hereinafter also designated as the “hydroxy mixed ethers”, the cleaning power of the enzyme-containing preparation can be surprisingly significantly improved, both in comparison with surfactant-free systems as well as in comparison with systems that comprise alternative non-ionic surfactants, for example from the group of the polyalkoxylated fatty alcohols.

Exemplary preferred liquid washing or cleaning agent preparations include—
a) 0.1 to 20 wt. % amylase preparation,
b) 5 to 50 wt. % protease preparation,
c) 10 to 50 wt. % 1,2-propylene glycol, and
d) 2 to 30 wt. % non-ionic surfactant according to the general formula

R¹—CH(OH)CH₂O-(AO)_{w}-(A′O)ₓ-(A″O)_{y}-(A′″O)_{z}—R²
wherein R¹ represents a straight chain or branched, saturated, mono- or polyunsaturated C₆₋₂₄ alkyl or alkenyl group; R² represents a linear or branched hydrocarbon group containing 2 to 26 carbon atoms; A, A′, A″ and A′″ independently represent —CH₂CH₂, —CH₂CH₂—CH₂, —CH₂—CH(CH₃), —CH₂—CH₂—CH₂—CH₂, —CH₂—CH(CH₃)—CH₂—, —CH₂—CH(CH₂—CH₃); and w, x, y and z stand for values between 0.5 and 120, wherein x, y and/or z can also be 0.

Further exemplary formulations for particularly preferred preparations are illustrated in the following table —

| | Formulation 1 | Formulation 2 | Formulation 3 | Formulation 4 | Formulation 5 |
|---|---|---|---|---|---|
| Ingredient | [wt. %] | [wt. %] | [wt. %] | [wt. %] | [wt. %] |
| Protease preparation | 5.0 to 50 | — | 5.0 to 50 | 7.0 to 45 | 10 to 40 |
| Amylase preparation | — | 0.1 to 20 | 0.1 to 20 | 0.2 to 15 | 1.0 to 12 |
| 1,2-propylene glycol | 5 to 60 | 5 to 60 | 5 to 60 | 10 to 50 | 15 to 45 |
| Niosurfactant¹ | 0.5 to 30 | 0.5 to 30 | 0.5 to 30 | 2.0 to 25 | 5.0 to 20 |
| Water | 5 to 35 | 5 to 35 | 5 to 35 | 10 to 25 | 5.0 to 20 |

¹Non-ionic surfactant of the general formula - R¹—CH(OH)CH₂O-(AO)_{w}-(AO)ₓ-(A″O)_{y}-(A′″O)_{z}—R²,

wherein R¹ represents a straight chain or branched, saturated or mono- or polyunsaturated C₆₋₂₄ alkyl or alkenyl group; R² represents a linear or branched hydrocarbon group containing 2 to 26 carbon atoms; A, A′, A″ and A′″ independently represent —CH₂CH₂, —CH₂CH₂—CH₂, —CH₂—CH(CH₃), —CH₂—CH₂—CH₂—CH₂, —CH₂—CH(CH₃)—CH₂—, or —CH₂—CH(CH₂—CH₃); and w, x, y and z represents values from 0.5 to 120, wherein x, y and/or z can also be 0.

Stability of enzymes contained in the washing or cleaning agent preparations according to the invention can be significantly improved by using these non-ionic surfactants having one or more free hydroxyl groups on one or both terminal alkyl groups.

The weight fraction of these non-ionic surfactants in preferred liquid washing or cleaning agent preparations is 0.5 to 30 wt. %, preferably 2.0 to 25 wt. % and in particular 5.0 to 20 wt. %, relative to total weight of the washing or cleaning agent preparation.

Such end-capped polyoxyalkylated non-ionic surfactants are particularly preferred according to the formula

R¹O[CH₂CH₂O]ₓCH₂CH(OH)R²

wherein R¹ represents linear or branched, saturated or unsaturated, aliphatic or aromatic hydrocarbon groups containing 2 to 30 carbon atoms, preferably containing 4 to 22 carbon atoms; a further linear or branched, saturated or unsaturated, aliphatic or aromatic hydrocarbon group R² containing 1 to 30 carbon atoms; and x represents values from 1 to 90, preferably values from 30 to 80, and especially values from 30 to 60.

Particularly preferred are surfactants according to the formula

R¹O[CH₂CH(CH₃)O]ₓ[CH₂CH₂O]_{y}CH₂CH(OH)R²

wherein R¹ represents a linear or branched aliphatic hydrocarbon group containing 4 to 18 carbon atoms or mixtures thereof; R² represents a linear or branched hydrocarbon group containing 2 to 26 carbon atoms or mixtures thereof; and x represents values from 0.5 to 1.5 and y represents a value of at least 15. These non-ionic surfactants include C₂₋₂₆ fatty alcohol-(PO)₁-(EO)₁₅₋₄₀-2-hydroxyalkyl ether, in particular, C₈₋₁₀ fatty alcohol-(PO)₁-(EO)₂₂-2-hydroxydecyl ether.

Further preferred end-capped poly(oxyalkylated) non-ionic surfactants are those of the formula

R¹O[CH₂CH₂O]ₓ[CH₂CH(R³)O]_{y}CH₂CH(OH)R²

wherein R¹ and R² independently represent linear or branched, saturated, mono- or polyunsaturated hydrocarbon groups containing 2 to 26 carbon atoms; R³ is —CH₃, —CH₂CH₃, —CH₂CH₂—CH₃, —CH(CH₃)₂, preferably —CH₃; and x and y independently represent values from 1 to 32, wherein surfactants with R³=—CH₃ and values for x of 15 to 32 and y of 0.5 and 1.5 are quite particularly preferred.

Further preferred suitable non-ionic surfactants include end-blocked poly(oxyalkylated) non-ionic surfactants of the formula

R¹O[CH₂CH(R³)O]ₓ[CH₂]ₖCH(OH)[CH₂]ⱼOR²

wherein R¹ and R² independently represent linear or branched, saturated, unsaturated, aliphatic or aromatic hydrocarbon groups containing 1 to 30 carbon atoms; R³ represents H or methyl, ethyl, n-propyl, isopropyl, n-butyl, 2-butyl or 2-methyl-2-butyl group; x represents values from 1 to 30; k and j represent values from 1 to 12, preferably from 1 to 5. Each R³ in the above formula R¹O[CH₂CH(R³)O]ₓ[CH₂]ₖCH(OH)[CH₂]ⱼOR² can differ for the case where x≧2. R¹ and R² are preferably linear or branched, saturated or unsaturated, aliphatic or aromatic hydrocarbon groups containing 6 to 22 carbon atoms, groups containing 8 to 18 carbon atoms being particularly preferred. H, —CH₃ or —CH₂CH₃ are particularly preferred for R³. Particularly preferred values for x are in the range from 1 to 20 and more particularly in the range from 6 to 15.

As described above, each R³ in the above formula can differ when x≧2. This means the alkylene oxide unit in the straight brackets can be varied. If, for example, x has a value of 3, then the substituent R³ may be selected to form ethylene oxide (R³═H) or propylene oxide (R³═CH₃) units, which may be joined together in any order, for example (EO)(PO)(EO), (EO)(EO)(PO), (EO)(EO)(EO), (PO)(EO)(PO), (PO)(PO)(EO) and (PO)(PO)(PO). The value 3 for x was selected by way of example and may easily be larger, the range of variation increasing with increasing x-values and including, for example, a large number of (EO) groups combined with a small number of (PO) groups or vice versa.

Particularly preferred end-capped poly(oxyalkylated) alcohols corresponding to the above formula have values of k=1 and j=1, so that the above formula can be simplified to R¹O[CH₂CH(R³)O]ₓCH₂CH(OH)CH₂OR². In this last formula, R¹, R² and R³ are as defined above and x stands for numbers from 1 to 30, preferably 1 to 20 and especially 6 to 18. Surfactants in which the substituents Wand R² have 9 to 14 carbon atoms, R³ stands for H and x assumes values of 6 to 15 are particularly preferred.

Finally, non-ionic surfactants according to the general formula

R¹—CH(OH)CH₂O-(AO)_{w}—R²

have proven to be particularly effective, wherein R¹ represents a straight chain or branched, saturated, mono- or polyunsaturated C₆₋₂₄ alkyl or alkenyl group; R² represents a linear or branched hydrocarbon group containing 2 to 26 carbon atoms; A represents CH₂CH₂, —CH₂CH₂—CH₂, or —CH₂—CH(CH₃); and w represents values from 1 to 120, preferably 10 to 80, particularly 20 to 40.

Such non-ionic surfactants include C₄₋₂₂ fatty alcohol-(PO)₁₀₋₈₀-2-hydroxyalkyl ethers, in particular, the C₈₋₁₂ fatty alcohol-(EO)₂₂-2-hydroxydecyl ethers and the C₄₋₂₂ fatty alcohol-(EO)₄₀₋₈₀-2-hydroxyalkyl ethers.

Accordingly, a preferred subject matter of the present application is liquid washing or cleaning agent preparations according to one of the preceding claims wherein the non-ionic surfactant has the general formula

R¹—CH(OH)CH₂O-(AO)_{w}—R²

wherein R¹ represents a straight chain or branched, saturated or mono- or polyunsaturated C₆₋₂₄ alkyl or alkenyl group; R² represents a linear or branched hydrocarbon group containing 2 to 26 carbon atoms; A represents CH₂CH₂, —CH₂CH₂—CH₂, or —CH₂—CH(CH₃); and w represents values from 1 to 120, preferably 10 to 80, and particularly 20 to 40.

In addition to the above described enzymes, solvents and non-ionic surfactants from the group of hydroxy mixed ethers, washing or cleaning agent preparations according to the invention can include other ingredients such as active substances, builders, bleaching agents, surfactants, washing- or cleaning-active polymers, enzymes, corrosion inhibitors, fragrances or colorants.

Contrary to common washing or cleaning agents, preferred washing or cleaning agent preparations according to the invention include these further ingredients only in minor amounts, because by reducing the weight fraction of these ingredients, both cleaning power and meterability of these compositions can be improved.

In particular, washing or cleaning agent preparations comprising less than 20 wt. %, preferably less than 10 wt. % and especially less than 5 wt. % builders are inventively preferred. In particular, washing or cleaning agent preparations that are free of builders are particularly preferred.

In addition, washing or cleaning agent preparations comprising less than 10 wt. %, preferably less than 5 wt. % and especially less than 2 wt. % bleaching agent are preferred. In particular, washing or cleaning agent preparations that are free of bleaching agents are particularly preferred.

Even when the above-mentioned additional washing- or cleaning-active ingredients are preferably included in only minor amounts in washing or cleaning agent preparations according to the invention (i.e., are directly blended with them), it is nevertheless desirable to prepare these additional ingredients together with the preparations according to the invention into a washing or cleaning agent. In this respect, one skilled in the art can draw on known manufactured types of combined products containing a liquid fraction, wherein combined products that have proven to be particularly suitable enable the common fabrication of two, three, four or more mutually separate liquid preparations.

Preferred liquid preparations include those prepared together with one, preferably two or three additional liquid washing or cleaning agent preparations into a combined product. These additional liquid washing or cleaning agent preparations have a different composition from the enzyme-containing washing or cleaning agent preparation according to the invention. The additional liquid washing or cleaning agent preparations are preferably free of bleaching agent and/or phosphate.

Some exemplary formulations for particularly preferred bleaching agent- and phosphate-free preparations are illustrated in the following table—

| | Combination product 1 | | Combination product 2 | | Combination product 3 | |
|---|---|---|---|---|---|---|
| | Chamber 1 | Chamber 2 | Chamber 1 | Chamber 2 | Chamber 1 | Chamber 2 |
| Ingredient | [wt. %] | [wt. %] | [wt. %] | [wt. %] | [wt. %] | [wt. %] |
| Protease preparation | 5.0 to 50 | — | 7.0 to 45 | — | 10 to 40 | — |
| Amylase preparation | 0.1 to 20 | — | 0.2 to 15 | — | 1.0 to 12 | — |
| 1,2-propyleneglycol | 5 to 60 | — | 10 to 50 | — | 15 to 45 | — |
| Nonionic surfactant ¹ | 0.5 to 30 | 0 to 10 | 2.0 to 25 | 0 to 10 | 5.0 to 20 | 0 to 10 |
| Water | 5 to 35 | 10 to 60 | 10 to 25 | 15 to 50 | 5.0 to 20 | 20 to 40 |
| Carbonate | — | 10 to 60 | — | 15 to 50 | — | 20 to 40 |
| Citrate | — | 2 to 50 | — | 5 to 40 | — | 10 to 30 |
| Complexant ² | — | 0.5 to 50 | — | 0.5 to 40 | — | 0.5 to 30 |
| Polymer ³ | — | 2 to 30 | — | 5 to 25 | — | 7 to 20 |
| Misc. | Add 100 | Add 100 | Add 100 | Add 100 | Add 100 | Add 100 |

¹ Nonionic surfactant of the general formula R¹—CH(OH)CH₂O-(AO)_{w}-(AO)ₓ-(A″O)_{y}-(A′″O)_{z}—R² wherein R¹ represents a straight chain or branched, saturated, mono- or polyunsaturated C₆₋₂₄ alkyl or alkenyl group; R² represents a linear or branched hydrocarbon group containing 2 to 26 carbon atoms; A, A′, A″ and A′″ independently represent —CH₂CH₂, —CH₂CH₂—CH₂, —CH₂—CH(CH₃), —CH₂—CH₂—CH₂—CH₂, —CH₂—CH(CH₃)—CH₂—, —CH₂—CH(CH₂—CH₃); and w, x, y and z represent values from 0.5 to 120, wherein x, y and/or z can also be 0.
² Sequestrants from the group comprising phosphonates, MGDA.
³ Polymers from the group of polyacrylate or polymethacrylate copolymers.

In addition to the described surfactants and enzymes, the above-described compositions according to the invention can further include other washing- or cleaning-active substances such as builders, polymers, glass corrosion inhibitors, corrosion inhibitors, fragrances and perfume carriers. In addition, bleaching agents and bleach activators can also be employed. These preferred ingredients are more closely described below.

Builders include in particular zeolites, silicates, carbonates and organic cobuilders.

Crystalline layer-forming silicates of the general formula NaMSiₓO₂ₓ₊₁. y H₂O are preferably employed, wherein M represents sodium or hydrogen, x is a number from 1.9 to 22, preferably 1.9 to 4, wherein particularly preferred values for x are 2, 3 or 4, and y stands for a number from 0 to 33, preferably from 0 to 20. The crystalline layer-forming silicates of the formula NaMSiₓO₂ₓ₊₁.y H₂O are marketed, for example, by Clariant GmbH (Germany) under the trade names Na-SKS. Examples of these silicates are Na-SKS-1 (Na₂Si₂₂O₄₅ x H₂O, Kenyait), Na-SKS-2 (Na₂Si₁₄O₂₉ x H₂O, Magadiit), Na-SKS-3 (Na₂Si₈O₁₇ x H₂O) and Na-SKS-4 (Na₂Si₄O₉ x H₂O, Makatit).

Crystalline, layered silicates of formula NaMSiₓO₂ₓ₊₁, wherein x is 2, are particularly suitable for the purposes of the present invention. Both β- and also δ-sodium disilicates Na₂Si₂O₅ y H₂O such as Na-SKS-5 (δ-Na₂Si₂O₅), Na-SKS-7 (β-Na₂Si₂O₅, Natrosilit), Na-SKS-9 (NaHSi₂O₅H₂O), Na-SKS-10 (NaHSi₂O₅ 3H₂O, Kanemit), Na-SKS-11 (t-Na₂Si₂O₅) and Na-SKS-13 (NaHSi₂O₅) are preferred, with Na-SKS-6 (δ-Na₂Si₂O₅) particularly preferred.

Washing or cleaning agents preferably comprise a content by weight of crystalline layered silicates of formula NaMSiₓO₂ₓ₊₁.y H₂O of 0.1 to 20 wt. %, preferably 0.2 to 15 wt. % and particularly 0.4 to 10 wt. %, each based on the total weight of the composition.

Other useful builders include amorphous sodium silicates with a modulus (Na₂O:SiO₂ ratio) of 1:2 to 1:3.3, preferably 1:2 to 1:2.8 and more preferably 1:2 to 1:2.6, which dissolve with a delay and exhibit secondary wash cycle properties. The delay in dissolution compared with conventional amorphous sodium silicates can be obtained in various ways, for example, by surface treatment, compounding, compressing/compacting or by over-drying. In the context of the present invention, the term “amorphous” is understood to encompass “X-ray amorphous”. In other words, the silicates do not produce any of the sharp X-ray reflexes typical of crystalline substances in X-ray diffraction experiments, but at best one or more maxima of the scattered X-radiation, which have a width of several degrees of the diffraction angle.

Alternatively, or in combination with the above-cited amorphous sodium silicates, X-ray amorphous silicates are employed whose silicate particles yield blurred or even sharp diffraction maxima in electron diffraction experiments. This can be interpreted to mean that the products have microcrystalline regions from about ten to about a few hundred nm in size, values of up to at most about 50 nm and especially up to at most about 20 nm being preferred. These types of X-ray amorphous silicates similarly exhibit a delayed dissolution in comparison with customary water glasses. Compacted/densified amorphous silicates, compounded amorphous silicates and over dried X-ray-amorphous silicates are particularly preferred.

Further useful builders include the alkalinity sources. Alkali metal hydroxides, alkali metal carbonates, alkali metal hydrogen carbonates, alkali metal sesquicarbonates, alkali silicates, alkali metal silicates and mixtures thereof are examples of alkalinity sources that can be used, the alkali carbonates being preferably used, especially sodium carbonate, sodium hydrogen carbonate or sodium sesquicarbonate in the context of this invention. A builder system comprising a mixture of tripolyphosphate and sodium carbonate is particularly preferred. A builder system comprising a mixture of tripolyphosphate and sodium carbonate and sodium disilicate is also particularly preferred. Because of their low chemical compatibility with ingredients typically found in washing and cleaning compositions versus other builders, alkali metal hydroxides are preferably incorporated only in low amounts, advantageously in amounts 10 wt. % or less, preferably 6 wt. % or less, particularly preferably 4 wt. % or less, and particularly 2 wt. % or less, each based on total weight of the washing or cleaning composition. Compositions comprising 0.5 wt. % or less based on total weight of the composition, in particular, no alkali metal hydroxide, are particularly preferred.

Organic co-builders include polycarboxylates/polycarboxylic acids, polymeric polycarboxylates, aspartic acid, polyacetals, dextrins, other organic co builders and phosphonates. These classes of substances are described below.

Useful organic builders include polycarboxylic acids that can be used in the form of free acid and/or their sodium salts. Polycarboxylic acids in this context are understood to be carboxylic acids that carry more than one acid function. These include citric acid, adipic acid, succinic acid, glutaric acid, malic acid, tartaric acid, maleic acid, fumaric acid, sugar acids, amino carboxylic acids, nitrilotriacetic acid (NTA), providing such use is not ecologically unsafe, and mixtures thereof. In addition to their building effect, free acids also typically have the property of an acidifying component and hence also serve to establish a relatively low and mild pH for washing and cleaning compositions. Citric acid, succinic acid, glutaric acid, adipic acid, gluconic acid and any mixtures thereof are particularly mentioned in this regard.

Other suitable builders include additional polymeric polycarboxylates, for example, the alkali metal salts of polyacrylic or polymethacrylic acid, such as those having a relative molecular weight of about 500 to about 70,000 g/mol.

Molecular weights mentioned in this specification for polymeric polycarboxylates are weight-average molecular weights M_{w} of the particular acid form, which were determined by gel permeation chromatography (GPC) using a UV detector. The measurement was carried out against an external polyacrylic acid standard, which provides realistic molecular weight values by virtue of its structural similarity to the polymers investigated. These values differ significantly from the molecular weights measured against polystyrene sulfonic acids as the standard. Molecular weights measured against polystyrene sulfonic acids are generally significantly higher than molecular weights mentioned in this specification.

Particularly suitable polymers include polyacrylates, which preferably have a molecular weight of about 2,000 to about 20,000 g/mol. Because of their superior solubility, preferred representatives of this group include the short-chain polyacrylates, which have molecular weights of about 2,000 to about 10,000 g/mol and, more particularly, about 3,000 to about 5,000 g/mol.

Further suitable copolymeric polycarboxylates include those of acrylic acid with methacrylic acid and of acrylic acid or methacrylic acid with maleic acid. Copolymers of acrylic acid with maleic acid, which comprise 50 to 90 wt. % acrylic acid and 50 to 10 wt. % maleic acid, have proven to be particularly suitable. Their relative molecular weight, based on free acids, generally ranges from 2,000 to 70,000 g/mol, preferably 20,000 to 50,000 g/mol and especially 30,000 to 40,000 g/mol.

In order to improve water solubility, the polymers can also comprise allylsulfonic acids such as allyloxybenzene sulfonic acid and methallyl sulfonic acid as monomers.

Particular preference is also given to biodegradable polymers comprising more than two different monomer units, monomeric examples of which include salts of acrylic acid and maleic acid, as well as vinyl alcohol or vinyl alcohol derivatives, or salts of acrylic acid and of 2-alkylallylsulfonic acid, and also sugar derivatives.

Other preferred copolymers include those preferably containing acrolein and acrylic acid/acrylic acid salts or acrolein and vinyl acetate as monomers.

Exemplary polymeric actives for water softening include polymers with sulfonic acid groups, which are especially preferably employed.

Particularly preferred suitable polymers comprising sulfonic acid groups include copolymers of unsaturated carboxylic acids, monomers comprising sulfonic acid groups and, optionally, further ionic or non-ionogenic monomers.

In the context of the present invention, unsaturated carboxylic acids of the formula—

R⁵(R⁶)C═C(R⁷)—X—SO₃H

wherein R⁵ to R⁷ independently represent —H, —CH₃, a linear or branched, saturated alkyl group containing 2 to 12 carbon atoms, a linear or branched, mono- or polyunsaturated alkenyl group containing 2 to 12 carbon atoms, with —NH₂, —OH or —COOH substituted alkyl or alkenyl groups as defined above or —COOH or —COOR⁴, wherein R⁴ is a saturated or unsaturated, linear or branched hydrocarbon group containing 1 to 12 carbon atoms; and X represents an optionally present spacer group chosen from —(CH₂)ₙ— with n=0 to 4, —COO—(CH₂)ₖ— with k=1 to 6, —C(O)—NH—C(CH₃)₂— and —C(O)—NH—CH(CH₂CH₃)—.

Among the unsaturated carboxylic acids corresponding to the above formula, acrylic acid (R¹═R²═R³═H), methacrylic acid (R¹═R²═H; R³═CH₃) and/or maleic acid (R¹═COOH; R²═R³═H) are particularly preferred.

Preferred monomers containing sulfonic acid groups include those of the formula—

R⁵(R⁶)C═C(R⁷)—X—SO₃H

wherein R⁵ to R⁷ independently represent —H, —CH₃, a linear or branched, saturated alkyl group containing 2 to 12 carbon atoms, a linear or branched, mono- or polyunsaturated alkenyl group containing 2 to 12 carbon atoms, with —NH₂, —OH or —COOH substituted alkyl or alkenyl groups as defined above or —COOH or —COOR⁴, wherein R⁴ is a saturated or unsaturated, linear or branched hydrocarbon group containing 1 to 12 carbon atoms; and X is an optionally present spacer group chosen from —(CH₂)ₙ— with n=0 to 4, —COO—(CH₂)ₖ— with k=1 to 6, —C(O)—NH—C(CH₃)₂— and —C(O)—NH—CH(CH₂CH₃)—.

Among these monomers, those preferred have the formulas —

H₂C═CH—X—SO₃H

H₂C═C(CH₃)—X—SO₃H

HO₃S—X—(R⁶)C═C(R⁷)—X—SO₃H

wherein R⁶ and R⁷ independently represent —H, —CH₃, —CH₂CH₃, —CH₂CH₂CH₃, or —CH(CH₃)₂; and X is an optionally present spacer group chosen from —(CH₂)ₙ— with n=0 to 4, —COO—(CH₂)ₖ— with k=1 to 6, —C(O)—NH—C(CH₃)₂— and —C(O)—NH—CH(CH₂CH₃)—.

Accordingly, particularly preferred sulfonic acid-containing monomers include 1-acrylamido-1-propanesulfonic acid, 2-acrylamido-2-propanesulfonic acid, 2-acrylamido-2-methyl-1-propanesulfonic acid, 2-methacrylamido-2-methyl-1-propanesulfonic acid, 3-methacrylamido-2-hydroxy-propanesulfonic acid, allylsulfonic acid, methallylsulfonic acid, allyloxybenzenesulfonic acid, methallyloxybenzenesulfonic acid, 2-hydroxy-3-(2-propenyloxy) propanesulfonic acid, 2-methyl-2-propene-1-sulfonic acid, styrene sulfonic acid, vinylsulfonic acid, 3-sulfopropyl acrylate, 3-sulfopropyl methacrylate, sulfomethylacrylamide, sulfomethylmethacrylamide and water-soluble salts of the cited acids.

Additional ionic or non-ionic monomers particularly include ethylenically unsaturated compounds. Preferably, the content of these additional ionic or non-ionic monomers in the added polymers is 20 wt. % or less, based on the polymer. Particularly preferred polymers for use consist solely of monomers of the formula R¹(R²)C═C(R³)COOH and monomers of the formula R⁵(R⁶)C═C(R⁷)—X—SO₃H.

In summary, copolymers of—
i) unsaturated carboxylic acids of the formula R¹(R²)C═C(R³)COOH in which R¹ to R³ independently of one another stand for —H, —CH₃, a linear or branched, saturated alkyl group containing 2 to 12 carbon atoms, alkyl or alkenyl groups substituted by —NH₂, —OH or —COOH as defined above or stand for —COOH or —COOR⁴, wherein R⁴ is a saturated or unsaturated, linear or branched hydrocarbon group containing 1 to 12 carbon atoms;
ii) sulfonic acid group-containing monomers of formula R⁵(R⁶)C═C(R⁷)—X—SO₃H in which R⁵ to R⁷ independently of one another stand for —H, —CH₃, a linear or branched, saturated alkyl group containing 2 to 12 carbon atoms carbon atoms, a linear or branched, mono- or polyunsaturated alkenyl group containing 2 to 12 carbon atoms with —NH₂, —OH or stand for —COOH substituted alkyl or alkenyl groups as defined above or for —COOH or —COOR⁴, wherein R⁴ is a saturated or unsaturated, linear or branched hydrocarbon group containing 1 to 12 carbon atoms, and X is an optionally present spacer group selected from —(CH₂)ₙ— with n=0 to 4, —COO—(CH₂)ₖ— with k=1 to 6, —C(O)—NH—C(CH₃)₂— and —C(O)—NH—CH(CH₂CH₃)—; and
iii) optionally, additional ionic or non-ionic monomers are particularly preferred.

Further particularly preferred copolymers include—
i) one or a plurality of unsaturated carboxylic acids from the group acrylic acid, methacrylic acid and/or maleic acid;
ii) one or more monomers containing sulfonic acid groups of the formulae—

H₂C═CH—X—SO₃H

H₂C═C(CH₃)—X—SO₃H

HO₃S—X—(R⁶)C═C(R⁷)—X—SO₃H,
wherein R⁶ and R⁷ independently represent —H, —CH₃, —CH₂CH₃, —CH₂CH₂CH₃, or —CH(CH₃)₂; and X is an optionally present spacer group chosen from —(CH₂)ₙ— with n=0 to 4, —COO—(CH₂)ₖ— with k=1 to 6, —C(O)—NH—C(CH₃)₂— and —C(O)—NH—CH(CH₂CH₃)—; and
iii) optional additional ionic or non-ionic monomers.

Copolymers can contain monomers from groups i) and ii) and optionally iii) in varying amounts, wherein all representatives of group i) can be combined with all representatives of group ii) and all representatives of group iii). Particularly preferred polymers have defined structural units, which are described below.

For example, preferred copolymers have structural units of the formula—

—[CH₂—CHCOOH]ₘ[CH₂—CHC(O)—Y—SO₃H]ₚ—

wherein m and p each represent a whole natural number from 1 to 2,000; and Y represents a spacer group chosen from substituted or unsubstituted aliphatic, aromatic or substituted aromatic hydrocarbon groups containing 1 to 24 carbon atoms, with spacer groups where Y represents —O—(CH₂)ₙ— with n=0 to 4, for —O—(C₆H₄)—, for —NH—C(CH₃)₂— or —NH—CH(CH₂CH₃)— being preferred.

These polymers are produced by copolymerization of acrylic acid with an acrylic acid derivative containing sulfonic acid groups. If the acrylic acid derivative containing sulfonic acid groups is copolymerized with methacrylic acid, then another polymer results whose incorporation is likewise preferred. Appropriate copolymers comprise structural units of the formula—

—[CH₂—C(CH₃)COOH]ₘ—[CH₂—CHC(O)—Y—SO₃H]ₚ—

wherein m and p each represent for a whole natural number from 1 to 2,000; and Y represents a spacer group chosen from substituted or unsubstituted aliphatic, aromatic or substituted aromatic hydrocarbon groups containing 1 to 24 carbon atoms, with spacer groups where Y represents —O—(CH₂)ₙ— with n=0 to 4, for —O—(C₆H₄)—, for —NH—C(CH₃)₂— or —NH—CH(CH₂CH₃)— being preferred.

Analogously, acrylic acid and/or methacrylic acid may also be copolymerized with methacrylic acid derivatives containing sulfonic acid groups so that the structural units in the molecule are changed. Consequently, copolymers that comprise structural units of the formula—

—[CH₂—CHCOOH]ₘ—[CH₂—C(CH₃)C(O)—Y—SO₃H]ₚ—

wherein m and p each represent a whole natural number from 1 to 2,000; and Y represents a spacer group chosen from substituted or unsubstituted aliphatic, aromatic or substituted aromatic hydrocarbon groups containing 1 to 24 carbon atoms, with spacer groups where Y represents —O—(CH₂)ₙ— with n=0 to 4, for —O—(C₆H₄)—, for —NH—C(CH₃)₂— or —NH—CH(CH₂CH₃)— being particularly preferred. Likewise, preferred copolymers comprise structural units of the formula—

[CH₂—C(CH₃)COOH]ₘ—[CH₂—C(CH₃)C(O)—Y—SO₃H]ₚ—

wherein m and p each represent a whole natural number from 1 to 2,000; and Y represents a spacer group chosen from substituted or unsubstituted aliphatic, aromatic or substituted aromatic hydrocarbon groups containing 1 to 24 carbon atoms, with spacer groups where Y represents —O—(CH₂)ₙ— with n=0 to 4, for —O—(C₆H₄)—, for —NH—C(CH₃)₂— or —NH—CH(CH₂CH₃)— being preferred.

Instead of or in addition to acrylic acid and/or methacrylic acid, maleic acid can also be incorporated as a particularly preferred monomer from group i). In this way, one arrives at inventively preferred copolymers comprising structural units of the formula—

—[HOOCCH—CHCOOH]ₘ—[CH₂—CHC(O)—Y—SO₃H]ₚ—

wherein m and p each represent a whole natural number from 1 to 2,000; and Y represents a spacer group selected from substituted or unsubstituted aliphatic, aromatic or araliphatic hydrocarbon groups containing 1 to 24 carbon atoms, with spacer groups where Y represents —O—(CH₂)ₙ— with n=0 to 4, for —O—(C₆H₄)—, for —NH—C(CH₃)₂— or —NH—CH(CH₂CH₃)— being preferred. In addition, copolymers are inventively preferred that comprise the structural units of formula—

—[HOOCCH—CHCOOH]ₘ—[CH₂—C(CH₃)C(O)O—Y—SO₃H]ₚ—

wherein m and p each represent a whole natural number from 1 to 2,000; and Y represents a spacer group selected from substituted or unsubstituted aliphatic, aromatic or substituted aromatic hydrocarbon groups containing 1 to 24 carbon atoms, with spacer groups where Y represents —O—(CH₂)ₙ— with n=0 to 4, for —O—(C₆H₄)—, for —NH—C(CH₃)₂— or —NH—CH(CH₂CH₃)— being preferred.

In summary, preferred copolymers comprise structural units of the formulae—

—[CH₂—CHCOOH]ₘ—[CH₂—CHC(O)—Y—SO₃H]ₚ—

—[CH₂—C(CH₃)COOH]ₘ—[CH₂—CHC(O)—Y—SO₃H]ₚ—

—[CF₁₂—CHCOOH]ₘ—[CH₂—C(CH₃)C(O)—Y—SO₃H]ₚ—

—[CH₂—C(CH₃)COOH]ₘ[CH₂—C(CH₃)C(O)—Y—SO₃H]ₚ—

—[HOOCCH—CHCOOH]ₘ—[CH₂—CHC(O)—Y—SO₃H]ₚ—

—[HOOCCH—CHCOOH]ₘ—[CH₂—C(CH₃)C(O)O—Y—SO₃H]ₚ—

wherein m and p represent a whole natural number from 1 to 2,000; and Y represents a spacer group chosen from substituted or unsubstituted aliphatic, aromatic or substituted aromatic hydrocarbon groups containing 1 to 24 carbon atoms, with spacer groups where Y represents —O—(CH₂)ₙ— with n=0 to 4, —O—(C₆H₄)—, —NH—C(CH₃)₂— or —NH—CH(CH₂CH₃)— being preferred.

Sulfonic acid groups may be present in the polymers in completely or partly neutralized form, meaning, the acidic hydrogen atom of the sulfonic acid groups are replaced by metal ions, preferably alkali metal ions and more particularly sodium ions, in some or all of the sulfonic acid groups. The addition of copolymers containing partly or fully neutralized sulfonic acid groups is preferred according to the invention.

Monomeric distribution of inventively preferred copolymers used ranges from, for copolymers that comprise only monomers defined in groups i) and ii), preferably 5 to 95 wt. % for i) and ii) respectively, particularly preferably 50 to 90 wt. % monomer from group i) and 10 to 50 wt. % monomer from group ii) respectively, based on the polymer.

Particularly preferred terpolymers include those having 20 to 85 wt. % monomer from group i), 10 to 60 wt. % monomer from group ii) and 5 to 30 wt. % monomer from group iii).

The molecular weight of the inventively preferred sulfo-copolymers used can be varied to adapt the properties of the polymer to the desired application requirement. Preferred washing or cleaning compositions are those wherein the molecular weights of the copolymers are about 2,000 to 200,000 g/mol, preferably 4,000 to 25,000 g/mol, and especially 5,000 to 15,000 g/mol.

In a further preferred embodiment, the inventive washing or cleaning agent preparations include a hydrophobically modified copolymer. Surprisingly, the cleaning power of the enzymes, particularly the proteases, is further improved by addition of hydrophobically modified copolymers. Particularly preferred hydrophobically modified copolymers contain—
i) monomers from the group of the mono- or polyunsaturated carboxylic acids;
ii) monomers of the general formula R¹(R²)C═C(R³)—X—R⁴, wherein R¹ to R³ independently represent —H, —CH₃ or —C₂H₅; X is an optionally present spacer group chosen from —CH₂—, —C(O)O— and —C(O)—NH—; and R⁴ represents a straight chain or branched saturated alkyl group containing 2 to 22 carbon atoms or an unsaturated, preferably aromatic group, containing 6 to 22 carbon atoms; and
iii) optionally, further monomers.

Consequently, a further preferred subject matter of the present application is liquid, aqueous washing or cleaning agent preparations containing—
a) at least one washing- or cleaning-active enzyme;
b) 1,2-propylene glycol;
c) non-ionic surfactant of the general formula

R¹—CH(OH)CH₂O-(AO)_{w}-(A′O)ₓ-(A″O)_{y}-(A′″O)_{z}—R²
wherein R¹ represents a straight chain or branched, saturated, mono- or polyunsaturated C₆₋₂₄ alkyl or alkenyl group; R² represents a linear or branched hydrocarbon group containing 2 to 26 carbon atoms; A, A′, A″ and A′″ independently represent —CH₂CH₂, —CH₂CH₂—CH₂, —CH₂—CH(CH₃), —CH₂—CH₂—CH₂—CH₂, —CH₂—CH(CH₃)—CH₂—, or —CH₂—CH(CH₂—CH₃); and w, x, y and z represent values from 0.5 to 120, wherein x, y and/or z can also be 0;
d) at least one copolymer containing
   i) monomers from the group of the mono- or polyunsaturated carboxylic acids; or
   ii) monomers of the general formula

R¹(R²)C═C(R³)—X—R⁴,
wherein R¹ to R³ independently represent —H, —CH₃ or —C₂H₅; X is an optionally present spacer group chosen from —CH₂—, —C(O)O— and —C(O)—NH—; and R⁴ represents a straight chain or branched saturated alkyl group containing 2 to 22 carbon atoms or an unsaturated, preferably aromatic group containing 6 to 22 carbon atoms; and
e) optionally, further monomers

Particularly preferred copolymers d) comprise as the carboxyl group-containing monomers i) acrylic acid, methacrylic acid, ethacrylic acid, α-chloroacrylic acid, α-cyanoacrylic acid, crotonic acid, α-phenylacrylic acid, maleic acid, maleic anhydride, fumaric acid, itaconic acid, citraconic acid, methylenemalonic acid, sorbic acid, cinnamic acid or their mixtures.

Particularly preferred copolymers d) comprise monomers of the general formula R¹(R²)C═C(R³)—X—R⁴ as an added non-ionic monomers ii). Particularly preferred monomers of this type include butene, isobutene, pentene, 3-methylbutene, 2-methylbutene, cyclopentene, hexene, 1-hexene, 2-methlypent-1-ene, 3-methylpent-1-ene, cyclohexene, methylcyclopentene, cycloheptene, methylcyclohexene, 2,4,4-trimethylpent-1-ene, 2,4,4-trimethylpent-2-ene, 2,3-dimethylhex-1-ene, 2,4-dimethylhex-1-ene, 2,5-dimethlyhex-1-ene, 3,5-dimethylhex-1-ene, 4,4-dimethylhex-1-ene, ethylcyclohexyne, 1-octene, α-olefins containing 10 or more carbon atoms such as for example 1-decene, 1-dodecene, 1-hexadecene, 1-octadecene and C22-α-olefin, 2-styrene, α-methylstyrene, 3-methylstyrene, 4-propylstyrene, 4-cyclohexylstyrene, 4-dodecylstyrene, 2-ethyl-4-benzylstyrene, 1-vinylnaphthalene, 2-vinylnaphthalene, methyl acrylate, ethyl acrylate, propyl acrylate, butyl acrylate, pentyl acrylate, hexyl acrylate, methyl methacrylate, N-(methyl)acrylamide, 2-ethylhexyl acrylate, 2-ethylhexyl methacrylate, N-(2-ethylhexyl)acrylamide, octyl acrylate, octyl methacrylate, N-(octyl)acrylamide, lauryl acrylate, lauryl methacrylate, N-(lauryl)acrylamide, stearyl acrylate, stearyl methacrylate, N-(stearyl)acrylamide, behenyl acrylate, behenyl methacrylate and N-(behenyl)acrylamide or their mixtures.

Exemplary formulations for particularly preferred bleaching agent- and phosphate-free combination products are illustrated in the following table—

| | Combination product 1 | | Combination product 2 | | Combination product 3 | |
|---|---|---|---|---|---|---|
| | Chamber 1 | Chamber 2 | Chamber 1 | Chamber 2 | Chamber 1 | Chamber 2 |
| Ingredient | [wt. %] | [wt. %] | [wt. %] | [wt. %] | [wt. %] | [wt. %] |
| Protease preparation | 5.0 to 50 | — | 7.0 to 45 | — | 10 to 40 | — |
| Amylase preparation | 0.1 to 20 | — | 0.2 to 15 | — | 1.0 to 12 | — |
| 1,2-propylene glycol | 5 to 60 | — | 10 to 50 | — | 15 to 45 | — |
| Nonionic surfactant ¹ | 0.5 to 30 | 0 to 10 | 2.0 to 25 | 0 to 10 | 5.0 to 20 | 0 to 10 |
| Water | 5 to 35 | 10 to 60 | 10 to 25 | 15 to 50 | 5.0 to 20 | 20 to 40 |
| Carbonate | — | 10 to 60 | — | 15 to 50 | — | 20 to 40 |
| Citrate | — | 2 to 50 | — | 5 to 40 | — | 10 to 30 |
| Complexant ² | — | 0.5 to 50 | — | 0.5 to 40 | — | 0.5 to 30 |
| Polymer ³ | — | 2 to 30 | — | 5 to 25 | — | 7 to 20 |
| Misc. | Add 100 | Add 100 | Add 100 | Add 100 | Add 100 | Add 100 |

¹ Nonionic surfactant of the general formula R¹—CH(OH)CH₂O-(AO)_{w}-(A^{′}O)ₓ-(A″O)_{y}-(A^{′″}O)_{z}—R², wherein R¹ is a straight chain or branched, saturated or mono- or polyunsaturated C₆₋₂₄ alkyl or alkenyl group; R² is a linear or branched hydrocarbon group containing 2 to 26 carbon atoms; A, A′, A″ and A′″ independently are —CH₂CH₂, —CH₂CH₂—CH₂, —CH₂—CH(CH₃), —CH₂—CH₂—CH₂—CH₂, —CH₂—CH(CH₃)—CH₂—, or —CH₂—CH(CH₂—CH₃); and w, x, y and z are values from 0.5 to 120, wherein x, y and/or z can also be 0.
² sequestrants from the group comprising phosphonates, MGDA
³ copolymers containing i) monomers from the group of the mono- or polyunsaturated carboxylic acids, or ii) monomers of the general formula R¹(R²)C═C(R³)—X—R⁴, wherein R¹ to R³ independently are —H, —CH₃ or —C₂H₅; X is an optionally present spacer group chosen from —CH₂—, —C(O)O— and —C(O)—NH—; and R⁴ is a straight chain or branched saturated alkyl group containing 2 to 22 carbon atoms or an unsaturated, preferably aromatic group containing 6 to 22 carbon atoms.

Similarly, other preferred builders include polymeric amino dicarboxylic acids, their salts or their precursors. Polyaspartic acids or their salts are particularly preferred.

Further suitable builders include polyacetals obtained by treating dialdehydes with polyol carboxylic acids possessing 5 to 7 carbon atoms and at least 3 hydroxyl groups. Preferred polyacetals are obtained from dialdehydes such as glyoxal, glutaraldehyde, or terephthalaldehyde, as well as their mixtures, and from polycarboxylic acids such as gluconic acid and/or glucoheptonic acid.

Further suitable organic builders include dextrins, for example, oligomers or polymers of carbohydrates obtained by partial hydrolysis of starches. The hydrolysis can be carried out using typical processes, for example, acidic or enzymatic catalyzed processes. The hydrolysis products preferably have average molecular weights in the range of 400 to 500,000 g/mol. A polysaccharide with a dextrose equivalent (DE) of 0.5 to 40, more particularly, 2 to 30 is preferred, DE being an accepted measure of the reducing effect of a polysaccharide in comparison with dextrose, which has a DE of 100. Both maltodextrins with a DE from 3 to 20 and dry glucose syrups with a DE from 20 to 37, as well as so-called yellow dextrins and white dextrins with relatively high molecular weights of 2,000 to 30,000 g/mol may be used.

Oxidized derivatives of such dextrins concern their reaction products with oxidizing agents capable of oxidizing at least one alcohol function of the saccharide ring to the carboxylic acid function.

Oxydisuccinates and other derivatives of disuccinates, preferably ethylenediamine disuccinate, are also further suitable cobuilders. Ethylenediamine-N,N″-disuccinate (EDDS) is preferably used here in the form of its sodium or magnesium salts. In this context, glycerine disuccinates and glycerine trisuccinates are also preferred. Suitable addition quantities are from 3 to 15 wt. %.

Automatic dishwashing agents according to the invention particularly preferably comprise methyl glycine diacetic acid or a salt of methyl glycine diacetic acid.

Other useful organic co-builders include acetylated hydroxycarboxylic acids and salts thereof, which optionally may also be present in lactone form and which contain at least 4 carbon atoms, at least one hydroxyl group and at most two acid groups.

In addition, any compounds capable of forming complexes with alkaline earth metal ions may be used as co-builders.

Surfactants include nonionic, anionic, cationic and amphoteric surfactants.

All nonionic surfactants known to one skilled in the art can be used. As additional nonionic surfactants, alkyl glycosides satisfying the general formula RO(G)ₓ can be added, wherein R is a primary linear or methyl-branched, particularly 2-methyl-branched, aliphatic group containing 8 to 22, preferably 12 to 18 carbon atoms; and G is a glycose unit containing 5 or 6 carbon atoms, preferably glucose. The degree of oligomerization x, which defines the distribution of monoglycosides and oligoglycosides, is any number from 1.0 to 10.0, preferably 1.2 to 1.4.

Another class of nonionic surfactants which may be used, either as the sole nonionic surfactant or in combination with other nonionic surfactants, are alkoxylated, preferably ethoxylated or ethoxylated and propoxylated, fatty acid alkyl esters preferably containing 1 to 4 carbon atoms in the alkyl chain.

Nonionic surfactants of the amine oxide type such as N-coco alkyl-N,N-dimethylamine oxide and N-tallow alkyl-N,N-dihydroxyethylamine oxide, and from fatty acid alkanolamides may also be suitable. The amount in which these nonionic surfactants are used is preferably no more than the amount in which the ethoxylated fatty alcohols are used and, particularly no more than half that amount.

Other suitable surfactants are polyhydroxy fatty acid amides corresponding to the formula—

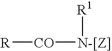

wherein R is an aliphatic acyl group containing 6 to 22 carbon atoms; R¹ is hydrogen, an alkyl or hydroxyalkyl group containing 1 to 4 carbon atoms; and [Z] is a linear or branched polyhydroxyalkyl group containing 3 to 10 carbon atoms and 3 to 10 hydroxyl groups. The polyhydroxyfatty acid amides are known substances, normally obtained by reductive amination of a reducing sugar with ammonia, an alkylamine or an alkanolamine and subsequent acylation with a fatty acid, a fatty acid alkyl ester or a fatty acid chloride.

Polyhydroxy fatty acid amides also includes compounds corresponding to the formula—

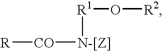

wherein R is a linear or branched alkyl or alkenyl group containing 7 to 12 carbon atoms; R¹ is a linear, branched or cyclic alkyl group or an aryl group containing 2 to 8 carbon atoms; R² is a linear, branched or cyclic alkyl group or an aryl group or an oxyalkyl group containing 1 to 8 carbon atoms, C₁₋₄-alkyl- or phenyl groups being preferred; and [Z] is a linear polyhydroxyalkyl group wherein the alkyl chain is substituted by at least two hydroxyl groups, or alkoxylated, preferably ethoxylated or propoxylated derivatives of that group.

[Z] is preferably obtained by reductive amination of a reducing sugar such as glucose, fructose, maltose, lactose, galactose, mannose or xylose. The N-alkoxy- or N-aryloxy-substituted compounds may then be converted into the required polyhydroxy fatty acid amides by reaction with fatty acid methyl esters in the presence of an alkoxide as catalyst.

Preferred surfactants include weakly foaming nonionic surfactants. Washing or cleaning compositions, particularly cleaning compositions for automatic dishwashers, are especially preferred when they include nonionic surfactants from the group of alkoxylated alcohols. Preferred nonionic surfactants include alkoxylated, advantageously ethoxylated, particularly primary alcohols preferably containing 8 to 18 carbon atoms and, on average, 1 to 12 moles of ethylene oxide (EO) per mole of alcohol, in which the alcohol group may be linear or, preferably, methyl-branched in the 2-position or may contain, for example, linear and methyl-branched groups in the form of mixtures typically present in oxo alcohol groups. Particularly preferred are alcohol ethoxylates with linear groups from alcohols of natural origin with 12 to 18 carbon atoms (e.g., from coco-, palm-, tallow- or oleyl alcohol), and an average of 2 to 8 EO per mole alcohol. Exemplary preferred ethoxylated alcohols include C₁₂₋₁₄ alcohols with 3 EO or 4 EO, C₉₋₁₁ alcohols with 7 EO, C₁₃₋₁₅ alcohols with 3 EU, 5 EO, 7 EO or 8 EO, C₁₂₋₁₈ alcohols with 3 EO, 5 EO or 7 EO and mixtures thereof, such as mixtures of C₁₂₋₁₄ alcohol with 3 EO and C₁₂₋₁₈ alcohol with 5 EO. The cited degrees of ethoxylation constitute statistically average values that can be a whole or a fractional number for a specific product. Preferred alcohol ethoxylates have a narrowed homolog distribution (narrow range ethoxylates, NRE). In addition to these non-ionic surfactants, fatty alcohols with more than 12 EO can also be used. Examples of these are tallow fatty alcohol with 14 EO, 25 EO, 30 EO or 40 EO.

Accordingly, ethoxylated nonionic surfactant(s) prepared from C₆₋₂₀ monohydroxy alkanols or C₆₋₂₀ alkyl phenols or C₁₆₋₂₀ fatty alcohols and more than 12 mole, preferably more than 15 mole and especially more than 20 mole ethylene oxide per mole alcohol, are used with particular preference. A particularly preferred nonionic surfactant is obtained from a straight-chain fatty alcohol containing 16 to 20 carbon atoms (C₁₆₋₂₀ alcohol), preferably a C₁₋₈ alcohol, and at least 12 moles, preferably at least 15 moles and more preferably at least 20 moles of ethylene oxide. Of these nonionic surfactants, the so-called “narrow range ethoxylates” are particularly preferred. Moreover, combinations of one or more tallow fat alcohols with 20 to 30 EO and silicone defoamers are particularly preferably used.

Nonionic surfactants having a melting point above room temperature can be used with particular preference. Nonionic surfactant(s) with a melting point above 20° C., preferably above 25° C., particularly preferably from 25 to 60° C., and especially from 26.6 to 43.3° C., is/are particularly preferred.

Suitable nonionic surfactants having a melting and/or softening point in the cited temperature range include weakly foaming nonionic surfactants that can be solid or highly viscous at room temperature. If nonionic surfactants are used that are highly viscous at room temperature, then it is preferred that they have a viscosity of at least 20 Pa s or greater, preferably at least 35 Pa s and especially at least 40 Pa s. Nonionic surfactants having a waxy consistency at room temperature are also preferred, depending on the application.

Nonionic surfactants from the group of alkoxylated alcohols, particularly preferably from the group of mixed alkoxylated alcohols, and especially from the group of EO-AO-EO nonionic surfactants are likewise incorporated with particular preference.

Preferably, the room temperature solid nonionic surfactant additionally has propylene oxide units in the molecule. These PO units preferably make up as much as 25% by weight, more preferably as much as 20% by weight and, especially up to 15% by weight of the total molecular weight of the nonionic surfactant. Particularly preferred nonionic surfactants include ethoxylated monohydroxyalkanols or alkylphenols having additional polyoxyethylene-polyoxypropylene block copolymer units. The alcohol or alkylphenol component of these nonionic surfactant molecules preferably makes up at least 30 wt. % or greater, more preferably at least 50 wt. % or greater, and most preferably at least 70 wt. % or greater of the total molecular weight of these nonionic surfactants. Preferred compositions comprise ethoxylated and propoxylated nonionic surfactants wherein the propylene oxide units in the molecule make up as much as 25% by weight, preferably as much as 20% by weight and, especially up to 15% by weight of the total molecular weight of the nonionic surfactant.

Useful preferred surfactants that are solid at room temperature include alkoxylated nonionic surfactants, more particularly, ethoxylated primary alcohols, and mixtures of these surfactants with structurally more complex surfactants, such as polyoxypropylene/polyoxyethylene/polyoxypropylene ((PO/EO/PO) surfactants). Such (PO/EO/PO) nonionic surfactants have good foam control.

Other particularly preferred nonionic surfactants with melting points above room temperature comprise 40 to 70% of a polyoxypropylene/polyoxyethylene/polyoxypropylene block polymer blend containing 75% by weight of an inverted block copolymer of polyoxyethylene and polyoxypropylene with 17 moles of ethylene oxide and 44 moles of propylene oxide and 25% by weight of a block copolymer of polyoxyethylene and polyoxypropylene initiated with trimethylolpropane and comprising 24 moles of ethylene oxide and 99 moles of propylene oxide per mole of trimethylolpropane.

Particularly preferred nonionic surfactants in the context of the present invention include weakly foaming non-ionic surfactants having alternating ethylene oxide and alkylene oxide units. Among these, surfactants with EO-AO-EO-AO blocks are again preferred, wherein one to ten EO or AO groups respectively are linked together before a block of the other groups follows. Here, nonionic surfactants of the general formula—

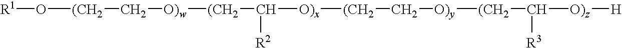

are preferred, wherein R¹ is a linear or branched, saturated or mono- or polyunsaturated C₆₋₂₄-alkyl or alkenyl group; R² and R³ independently are —CH₃, —CH₂CH₃, —CH₂CH₂—CH₃, or —CH(CH₃)₂; and w, x, y, z independently of one another are whole numbers from 1 to 6.

Preferred nonionic surfactants of the previous formula can be manufactured by known methods from the corresponding alcohols R¹—OH and ethylene- or alkylene oxide. The group R¹ in the previous formula can vary depending on the origin of the alcohol. When natural sources are used, the group R¹ has an even number of carbon atoms and generally is not branched, the linear alcohols of natural origin containing 12 to 18 carbon atoms (e.g., coconut, palm, tallow or oleyl alcohol) being preferred. Alcohols available from synthetic sources include Guerbet alcohols or mixtures of groups that are methyl branched in the 2-position or linear and methyl branched groups, as are typically present in oxo alcohols. Regardless of the type of alcohol used for manufacture of the nonionic surfactants comprised in the compositions, nonionic surfactants are preferred wherein R¹ in the previous formula is an alkyl group containing 6 to 24, preferably 8 to 20, particularly preferably 9 to 15 and particularly 9 to 11 carbon atoms.

In addition to propylene oxide, butylene oxide can be the alkylene oxide unit that alternates with the ethylene oxide unit in the preferred nonionic surfactants. However, other alkylene oxides are also suitable wherein R² or R³ independently are —CH₂CH₂—CH₃ or —CH(CH₃)₂. Preferably, nonionic surfactants of the previous formula are used wherein R² or R³ are —CH₃; w and x independently are values of 3 or 4; and y and z independently are values of 1 or 2.

In summary, nonionic surfactants are especially preferred that have a C₉₋₁₅ alkyl group with 1 to 4 ethylene oxide units, followed by 1 to 4 propylene oxide units, followed by 1 to 4 ethylene oxide units, followed by 1 to 4 propylene oxide units. These surfactants exhibit the required low viscosity in aqueous solution and, according to the invention, are used with particular preference.

The cited carbon chain lengths and degrees of ethoxylation or alkoxylation of the abovementioned nonionic surfactants represent statistically average values that can be a whole or fractional number for a specific product. Due to the manufacturing processes, commercial products of the cited formulae do not consist as a sole representative but rather are a mixture, wherein not only the carbon chain lengths but also the degrees of ethoxylation or alkoxylation can be average values and thus fractional numbers.

Of course, the abovementioned nonionic surfactants are not only be employed as single substances, but also as surfactant mixtures of two, three, four or more surfactants. Accordingly, surfactant mixtures do not refer to mixtures of nonionic surfactants that as a whole fall under one of the above cited general formulas, but rather refer to such mixtures that comprise two, three, four or more nonionic surfactants described by the different abovementioned general formulae.

When anionic surfactants are used as components of automatic dishwashing agents, their content, based on the total weight of the composition, is advantageously 4% or less by weight, preferably 2% or less by weight, and quite particularly preferably 1% or less by weight. Automatic dishwashing agents comprising no anionic surfactants are particularly preferred.

Cationic and/or amphoteric surfactants can be added instead of or in combination with the cited surfactants.

As cationic active substances, cationic compounds of the following formulae can be incorporated—

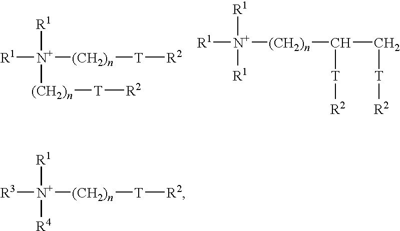

wherein each group R¹ independently is C₁₋₆ alkyl, -alkenyl or -hydroxyalkyl groups; each group R² independently is C₈₋₂₈ alkyl or -alkenyl groups; R³ is R¹ or (CH₂)ₙ-T-R²; R⁴ is R¹ or R² or (CH₂)ₙ-T-R²; T is —CH₂—, —O—CO— or —CO—O—; and n is an integer from 0 to 5.

In automatic dishwashing agents, cationic and/or amphoteric surfactant content is advantageously 6% or less by weight, preferably 4% or less by weight, quite particularly preferably 2% or less by weight, and in particular 1% or less by weight. Automatic dishwashing agents having no cationic or amphoteric surfactants are particularly preferred.

The group of the polymers includes, in particular the washing- or cleaning-active polymers, for example, the rinsing polymers and/or polymers active for water softening. Generally, in addition to non-ionic polymers, also cationic, anionic or amphoteric polymers are suitable for incorporation in washing or cleaning compositions.

In the context of the present application, “cationic polymers” include polymers carrying a positive charge in the polymer molecule. This can be realized, for example, by the presence of (alkyl)ammonium groups in the polymer chain or by other positively charged groups. Particularly preferred cationic polymers include quaternized cellulose derivatives, polysiloxanes containing quaternized groups, cationic guar derivatives, polymeric dimethyldiallylammonium salts and their copolymers with esters and amides of acrylic acid and methacrylic acid, copolymers of vinyl pyrrolidone with quaternized derivatives of dialkylamino acrylate and dialkylamino methacrylate, vinyl pyrrolidone/methoimidazolinium chloride copolymers, quaternized polyvinyl alcohols, or polymers listed under the INCI names Polyquaternium 2, Polyquaternium 17, Polyquaternium 18 and Polyquaternium 27.

In the context of the present invention, “amphoteric polymers” include polymers having, in addition to a positively charged group in the polymer chain, further negatively charged groups or monomer units. These groups include carboxylic acids, sulfonic acids or phosphonic acids.

Preferred washing or cleaning agents, in particular, preferred automatic dishwashing agents include those comprising a polymer a) possessing monomer units of the formula R¹R²C═CR³R⁴, wherein each group R¹, R², R³, R⁴ independently is hydrogen, derivatized hydroxyl groups, C₁₋₃₀ linear or branched alkyl groups, aryl, aryl substituted C₁₋₃₀ linear or branched alkyl groups, polyalkoxylated alkyl groups, heteroatomic organic groups having at least one positive charge without charged nitrogen, at least one quaternized nitrogen atom or at least one amino group with a positive charge in the pH range 2 to 11, or salts thereof, with the proviso that at least one group R¹, R², R³, R⁴ is a heteroatomic organic group with at least one positive charge without charged nitrogen, at least one quaternized nitrogen atom or at least one amino group with a positive charge. In the scope of the present application, particularly preferred cationic or amphoteric polymers comprise as the monomer unit a compound of the general formula—

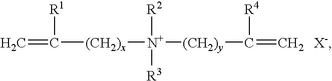

wherein R¹ and R⁴ independently are a linear or branched hydrocarbon group with 1 to 6 carbon atoms; R² and R³ independently are an alkyl, hydroxyalkyl or aminoalkyl group, wherein the alkyl group is linear or branched and has 1 to 6 carbon atoms, wherein it is preferably a methyl group; x and y independently are whole numbers from 1 to 3; and X⁻ represents a counter ion, preferably chloride, bromide, iodide, sulfate, hydrogen sulfate, methosulfate, lauryl sulfate, dodecylbenzene sulfonate, p-toluene sulfonate (tosylate), cumene sulfonate, xylene sulfonate, phosphate, citrate, formate, acetate or mixtures thereof.

Preferred groups R¹ and R⁴ in the above formula include —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, and —(CH₂CH₂—O)ₙH.

Quite particularly preferred polymers include those having a cationic monomer unit of the above general formula wherein R¹ and R⁴ are H; R² and R³ are methyl; and x and y are each 1. Monomer units corresponding to the formula—

H₂C═CH—(CH₂)—N⁺(CH₃)₂—(CH₂)—CH═CH₂X⁻

are also called DADMAC (diallyldimethylammonium chloride) where X⁻is chloride.

Further particularly preferred cationic or amphoteric polymers include a monomer unit of the general formula—

R¹HC═CR²—C(O)—NH—(CH₂)—N⁺R³R⁴R⁵X⁻

wherein R¹, R², R³, R⁴ and R⁵ independently are a linear or branched, saturated or unsaturated alkyl, or hydroxyalkyl group containing 1 to 6 carbon atoms, preferably a linear or branched alkyl group chosen from —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, and —(CH₂CH₂—O)ₙH; and x is a whole number from 1 to 6.

In the context of the present application, quite particularly preferred polymers possess a cationic monomer unit of the above general formula wherein R¹ is H; R², R³, R⁴ and R⁵ are methyl; and x is 3. The monomer units corresponding to the formula—

H₂C═C(CHS)—C(O)—NH—(CH₂)ₓ—N⁺(CH₃)₃X⁻

are also designated as MAPTAC (methylacrylamidopropyl-trimethyl ammonium chloride) when X⁻ is chloride.

According to the invention, preferred polymers include diallyldimethylammonium salts and/or acrylamidopropyl trimethylammonium salts as monomer units.

The previously mentioned amphoteric polymers possess not only cationic groups but also anionic groups or monomer units. These anionic monomer units originate, for example, from linear or branched, saturated or unsaturated carboxylates, linear or branched, saturated or unsaturated phosphonates, linear or branched, saturated or unsaturated sulfates or linear or branched, saturated or unsaturated sulfonates. Preferred monomer units include acrylic acid, (meth)acrylic acid, (dimethyl)acrylic acid, (ethyl)acrylic acid, cyanoacrylic acid, vinylacetic acid, allylacetic acid, crotonic acid, maleic acid, fumaric acid, cinnamic acid and its derivatives, allylsulfonic acids such as allyloxybenzene sulfonic acid and methallylsulfonic acid, or the allylphosphonic acids.

Preferred useful amphoteric polymers include alkylacrylamide/acrylic acid copolymers, alkylacrylamide/methacrylic acid copolymers, alkylacrylamide/methylmethacrylic acid copolymers, alkylacrylamide/acrylic acid/alkyl-aminoalkyl(meth)acrylic acid copolymers, alkylacrylamide/methacrylic acid/alkylaminoalkyl (meth)acrylic acid copolymers, alkylacrylamide/methylmethacrylic acid/alkylaminoalkyl (meth)acrylic acid copolymers, alkylacrylamide/alkyl methacrylate/alkylaminoethyl methacrylate/alkyl methacrylate copolymers, as well as copolymers of unsaturated carboxylic acids, cationically derivatized unsaturated carboxylic acids, and optionally additional ionic or nonionic monomers.

Preferred useful zwitterionic polymers include acrylamidoalkyltrialkylammonium chloride/acrylic acid copolymers as well as their alkali metal- and ammonium salts, acrylamidoalkyltrialkylammonium chloride/methacrylic acid copolymers as well as their alkali metal- and ammonium salts, and methacroylethylbetaine/methacrylate copolymers.

In addition, preferred amphoteric polymers include methacrylamidoalkyl-trialkylammonium chloride and dimethyl(diallyl)ammonium chloride as the cationic monomer in addition to one or more anionic monomers.

Particularly preferred amphoteric polymers include methacrylamidoalkyl-trialkylammonium chloride/dimethyl(diallyl)ammonium chloride/acrylic acid copolymers, methacrylamidoalkyltrialkylammonium chloride/dimethyl(diallyl)ammonium chloride/methacrylic acid copolymers, and methacrylamidoalkyltrialkylammonium chloride/dimethyl(diallyl)ammonium chloride/alkyl(meth)acrylic acid copolymers, as well as their alkali metal and ammonium salts.

Particularly preferred amphoteric polymers include methacrylamidopropyltrimethylammonium chloride/dimethyl(diallyl)ammonium chloride/acrylic acid copolymers, methacrylamidopropyltrimethylammonium chloride/dimethyl(diallyl)ammonium chloride/acrylic acid copolymers, and methacrylamidopropyltrimethylammonium chloride/dimethyl(diallyl)ammonium chloride/alkyl(meth)acrylic acid copolymers, as well as their alkali metal and ammonium salts.

In a particularly preferred embodiment of the present invention, the polymers are in preconditioned form. Suitable preconditioning of the polymers includes—
Encapsulation of the polymers by water-soluble or water-dispersible coating agents, preferably by water-soluble or water-dispersible natural or synthetic polymers;
Encapsulation of the polymers by water-insoluble, meltable coating agents, preferably by water-insoluble coating agents from waxes or paraffins having a melting point above 30° C.; and
Cogranulation of the polymers with inert carriers, preferably with carriers from washing- or cleaning-active substances, particularly preferably from builders or cobuilders.

Washing or cleaning compositions preferably comprise the abovementioned cationic and/or amphoteric polymers in amounts from 0.01 to 10 wt. %, based on total weight of the washing or cleaning composition. However, in the context of the present application, those washing or cleaning compositions are preferred in which the weight content of the cationic and/or amphoteric polymers is from 0.01 to 8 wt. %, preferably from 0.01 to 6 wt. %, preferably from 0.01 to 4 wt. %, particularly preferably from 0.01 to 2 wt. % and especially from 0.01 to 1 wt. %, each based on total weight of the automatic dishwashing agent.

Glass corrosion inhibitors prevent the occurrence of smears, streaks and scratches as well as iridescence on the surfaces of glasses washed in an automatic dishwasher. Preferred glass corrosion inhibitors include magnesium salts and zinc salts and magnesium complexes and zinc complexes.

Inventively preferred zinc salts, preferably of organic acids, particularly preferably of organic carboxylic acids, range from salts that are only soluble with difficulty or insoluble in water (i.e., with a solubility of 100 mg/l or less, preferably 10 mg/l or less, or especially 0.01 mg/l or less), to salts with solubilities in water 100 mg/l or greater, preferably 500 mg/l or greater, particularly preferably 1 g/l or greater, and especially 5 g/l or greater (all solubilities at water temperature of 20° C.). Slightly soluble zinc salts include zinc citrate, zinc oleate and zinc stearate, and soluble zinc salts include zinc formate, zinc acetate, zinc lactate and zinc gluconate.

A particular advantageous glass corrosion inhibitor employs at least one zinc salt of an organic carboxylic acid, particularly preferably zinc stearate, zinc oleate, zinc gluconate, zinc acetate, zinc lactate and/or zinc citrate. Zinc ricinoleate, zinc abietate and zinc oxalate are also preferred.

Corrosion inhibitors protect the tableware or the machine, with silver protection agents being particularly important in automatic dishwashing. Substances known from the art can be incorporated. Above all, silver protectors chosen from triazoles, benzotriazoles, bis-benzotriazoles, aminotriazoles, alkylaminotriazoles and transition metal salts or transition metal complexes may generally be used. Benzotriazole and/or alkylaminotriazole are particularly preferably used. Use of 3-amino-5-alkyl-1,2,4-triazoles or their physiologically compatible salts is inventively preferred. Preferred acids for salt formation include hydrochloric acid, sulfuric acid, phosphoric acid, carbonic acid, sulfurous acid, and organic carboxylic acids such as acetic acid, glycolic acid, citric acid and succinic acid. 5-Pentyl-, 5-heptyl-, 5-nonyl-, 5-undecyl-, 5-isononyl-, 5-versatic-10-acid alkyl-3-amino-1,2,4-triazoles as well as mixtures of these substances are quite particularly efficient.

In the context of the present invention, useful perfume oils or fragrances include individual odoriferous compounds such as synthetic products of the ester, ether, aldehyde, ketone, alcohol and hydrocarbon type. However, mixtures of various odoriferous substances, which together produce an attractive fragrant note, are preferably used. Perfume oils such as these may also contain natural odoriferous mixtures obtainable from vegetal sources (e.g., pine, citrus, jasmine, patchouli, rose or ylang-ylang oil).

Volatility of an odoriferous substance is crucial for its perceptibility. In addition to the nature of the functional groups and the structure of the chemical compound, molecular weight also plays an important role. Thus, most odoriferous substances have molecular weights of up to about 200 Daltons, with molecular weights of 300 Daltons and greater being quite an exception. Due to the different volatilities of odoriferous materials, the smell of a perfume or fragrance composed of a plurality of odoriferous substances changes in the course of evaporation, with the impressions of odor being subdivided into the “top note”, “middle note” or “body” and “end note” or “dry out”. As the perception of smell also depends to a large extent on the intensity of the odor, the top note of a perfume or fragrance consists not solely of highly volatile compounds, whereas the endnote consists to a large extent of less volatile, tenacious odoriferous substances. In perfume compositions, odoriferous substances of higher volatility can be bound onto particular fixatives whereby their rapid evaporation is impeded. In the following subdivision of odoriferous substances into “more volatile” or “tenacious” odoriferous substances, nothing is mentioned about odor impression and further, whether the relevant odoriferous substance is perceived as the top note or body note.

Fragrances may be directly incorporated, although it can also be advantageous to deposit the fragrances on carriers so that a long lasting fragrance is ensured due to slower fragrance release. Suitable carrier materials include cyclodextrins, with the cyclodextrin/perfume complexes optionally being coated with other auxiliaries.

Preferred colorants have high storage stability, are not affected by the other ingredients of the agent or by light, and do not have any pronounced adherence to substrates such as glass, ceramics or plastic dishes being treated with the colorant-containing agent, so as not to color them.

Another subject matter of this application is a process for washing dishes in an automatic dishwasher by using a liquid washing or cleaning agent preparation containing—

Washing or cleaning agent preparations according to the invention which are particularly preferably employed in these processes correspond to the compositions described above in detail. In order to avoid repetition, reference is made to the above embodiments. Preferred processes for cleaning tableware are those wherein the liquid washing or cleaning agent preparation is metered into the interior of the automatic dishwasher from a storage reservoir located in the automatic dishwasher and which comprises the multiple amounts of the washing or cleaning agent preparation needed for carrying out a cleaning process.

As explained in the introduction, the storage reservoir used for metering can be a storage reservoir integrated into the automatic dishwasher (i.e., a storage reservoir permanently fixed (built in) to the automatic dishwasher), and can also be an autarkic (i.e., an independent storage reservoir that can be inserted into the interior of the automatic dishwasher).

An example of an integrated storage reservoir is a receptacle built into the door of the automatic dishwasher and connected to the interior of the automatic dishwasher by supply line.

An example of an autarkic storage reservoir is a “top-down bottle” having a base outlet valve, and which can be placed, for example, in the cutlery basket of the automatic dishwasher.

The storage reservoir has at least one chamber for receiving a liquid washing or cleaning agent preparation according to the invention. In a preferred embodiment, the storage reservoir has more than one, preferably two, three, four or more separate chambers that are separated from each other, of which at least one chamber contains the liquid washing or cleaning agent preparations according to the invention and at least one, preferably at least two additional chambers, contain(s) liquid preparations having a different composition from that of the liquid washing or cleaning agent preparations according to the invention.

In particular, particularly preferred processes according to the invention use a storage reservoir having two separate chambers separated from one another, with one chamber containing a liquid washing or cleaning agent preparation according to the invention and the second chamber containing a different liquid, bleaching agent-free preparation.

In preferred cleaning processes a quantity of from 1.0 to 15 ml, preferably from 2.0 to 12 ml, and especially from 4.0 to 10 ml of the liquid washing or cleaning agent preparation according to the invention is metered per wash cycle into the interior of the automatic dishwasher.

The volume of preferred storage reservoirs containing one or more chambers is from 10 to 1000 ml, preferably from 20 to 800 ml, and especially from 50 to 500 ml.

As explained above, washing or cleaning agent preparations according to the invention have a particular temperature stability and are employed in the process according to the invention, in particular, for repeated metering of these preparations from the storage reservoirs located in the interior of the automatic dishwasher. Preferred processes according to the invention are those wherein the liquid washing or cleaning agent preparation, prior to being metered into the interior of the automatic dishwasher, remains in the storage reservoir that is located in the automatic dishwasher for at least two, preferably at least four, particularly preferably at least eight and in particular at least twelve separate cleaning processes.

In the context of the present application, “separate cleaning processes” are completed cleaning processes that preferably also include a pre-rinse cycle and/or a rinse cycle in addition to the main cleaning cycle, and which can be selected and actuated by means of the program switch of the automatic dishwasher. The duration of these separate cleaning processes is advantageously at least 15 minutes, advantageously from 20 to 360 minutes, preferably from 30 to 240 minutes.

The length of time between two separate cleaning processes within which the liquid washing or cleaning agent preparation is metered into the interior of the automatic dishwasher, is at least 20 minutes, preferably at least 60 minutes, particularly preferably at least 120 minutes.

Exposure to high temperatures by liquid washing or cleaning agent preparations according to the invention can vary widely during the processes according to the invention, wherein the liquid washing or cleaning agent preparations are particularly suitable for those processes in which the liquid washing or cleaning agent preparation in the storage reservoir is heated at least two times, preferably at least four times, particularly preferably at least eight times and in particular at least twelve times to temperatures of 30° C. or greater, preferably of 40° C. or greater, and particularly preferably of 50° C. or greater. Naturally, heating the liquid washing or cleaning agent preparation to temperatures above 60° C. or 70° C. or heating it twenty or thirty times can also be realized according to the invention.

In other words, in each of the sequential separate cleaning processes, the rinse liquor surrounding this storage reservoir heats the liquid washing or cleaning agent preparation in the storage reservoir. In preferred processes, liquid washing or cleaning agent preparation in the storage reservoir cools down between each cleaning processes to temperatures of 30° C. or less, preferably of 26° C. or less, and especially of 22° C. or less.

Use of nonionic surfactants of the general formula—

R¹—CH(OH)CH₂O-(AO)_{w}-(A′O)ₓ-(A″O)_{y}-(A′″O)_{z}—R²

wherein
R¹ is a straight chain or branched, saturated or mono- or polyunsaturated C₆₋₂₄ alkyl or alkenyl group;
R² is a linear or branched hydrocarbon group containing 2 to 26 carbon atoms;
A, A′, A″ and A′″ independently are —CH₂CH₂, —CH₂CH₂—CH₂, —CH₂—CH(CH₃), —CH₂—CH₂—CH₂—CH₂, —CH₂—CH(CH₃)—CH₂—, or —CH₂—CH(CH₂—CH₃); and
w, x, y and z stand for values between 0.5 and 120, wherein x, y and/or z can also be 0,
   in order to stabilize liquid enzyme preparations is another subject matter of the present application.

Preferably, nonionic surfactants of the general formula—

R¹—CH(OH)CH₂O-(AO)_{w}-(A′O)ₓ-(A″O)_{y}-(A″O)_{z}—R²

are employed in order to stabilize liquid preparations comprising amylase(s) and/or protease(s).

Particularly preferred stabilized washing or cleaning agent preparations and particularly preferred hydroxy mixed ethers employed for stabilization are explained in detail further above. In order to avoid repetition here, reference is made to the above embodiments.

### EXAMPLES

The following three cleaning agent formulations were prepared—

| | Alkaline | V1 | E1 |
|---|---|---|---|
| | cleaner [wt. %] | [wt. %] | [wt. %] |
| Carbonate | 27 | — | — |
| Citrate | 16 | — | — |
| MGDA | 16 | — | — |
| Polycarboxylate | 12 | — | — |
| Phosphonate | 2 | — | — |
| Water | 25 | 29 | 24 |
| Protease preparation | | 20 | 17 |
| Amylase preparation | | 7 | 6 |
| 1,2-propylene glycol | | 44 | 37 |
| Hydroxy mixed ether¹ | | — | 16 |
| Misc. | Add 100 | — | — |

These three cleaning agents were placed in separate closed and watertight pressure-equalized containers in a continuously working domestic automatic dishwasher (Miele G1220 Konti) for A period of ten cleaning cycles (intensive 75° C.).

Enzyme-containing compositions V1 (non-inventive) and E1 (inventive) were then combined with the alkaline cleaner, and the cleaning power of the resulting mixture was determined by the IKW method.

The following results (10=complete cleaning; 0=no cleaning) are shown for the stains egg yolk, milk, minced meat, intractable minced meat and oat flakes:

| | Alkaline cleaner + V1 | Alkaline cleaner + E1 |
|---|---|---|
| Egg yolk | 4.5 | 5.5 |
| Milk | 5.0 | 6.5 |
| Minced meat | 5.0 | 9.0 |
| Intractable minced meat | 4.5 | 5.0 |
| Oat flakes | 8.0 | 9.0 |

As the experimental results demonstrate, the enzyme-containing cleaner that was stabilized by addition of a hydroxy mixed ether exhibited significantly better cleaning results both on stains relevant to the amylase, as well as on those relevant to the protease than did the hydroxy mixed ether-free cleaner.

We claim:

1. Liquid, aqueous washing or cleaning agent preparation comprising:
a) at least one washing- or cleaning-active enzyme;
b) 1,2-propylene glycol; and
c) a nonionic surfactant of the general formula
R¹—CH(OH)CH₂O-(AO)_{w}-(A′O)ₓ-(A″O)_{y}-(A′″O)_{z}—R²,
wherein R¹ is a straight chain or branched, saturated or mono- or polyunsaturated C₆₋₂₄ alkyl or alkenyl group; R² is a linear or branched hydrocarbon group containing 2 to 26 carbon atoms; A, A′, A″ and A′″ independently are —CH₂CH₂, —CH₂CH₂—CH₂, —CF₁₂—CH(CH₃), —CH₂—CH₂—CH₂—CH₂, —CH₂—CH(CH₃)—CH₂—, or —CH₂—CH(CH₂—CH₃); and w, x, y and z are independently values from 0.5 to 120, wherein x, y and/or z can also be 0.

2. Liquid washing or cleaning agent preparation according to claim 1, wherein the washing- or cleaning-active enzyme is an amylase and/or protease.

3. Liquid washing or cleaning agent preparation according to claim 2, wherein the washing- or cleaning-active enzyme is at least an amylase present in an amount of 0.1 to 20 wt. %, based on total weight of the washing or cleaning agent preparation.

4. Liquid washing or cleaning agent preparation according to claim 2, wherein the washing- or cleaning-active enzyme is at least a protease present in an amount of 5 to 50 wt. %, based on total weight of the washing or cleaning agent preparation.

5. Liquid washing or cleaning agent preparation according to claim 1, wherein the 1,2-propylene glycol is present in an amount of 5 to 60 wt. %, based on total weight of the washing or cleaning agent preparation.

6. Liquid washing or cleaning agent preparation according to claim 1, wherein the nonionic surfactant has the general formula R¹—CH(OH)CH₂O-(AO)_{w}—R², wherein R¹ is a straight chain or branched, saturated or mono- or polyunsaturated C₆₋₂₄ alkyl or alkenyl group; R² is a linear or branched hydrocarbon group containing 2 to 26 carbon atoms; A is CH₂CH₂, —CH₂CH₂—CH₂, or —CH₂—CH(CH₃); and w is a value from 1 to 120.

7. Liquid washing or cleaning agent preparation according to claim 1, wherein the nonionic surfactant is present in an amount of 0.5 to 30 wt. %, based on total weight of the washing or cleaning agent preparation.

8. Liquid washing or cleaning agent preparation according to claim 1 further comprising water in an amount of 5 to 35 wt. %, based on total weight of the washing or cleaning agent preparation.

9. Liquid washing or cleaning agent preparation comprising:
a) 0.1 to 20 wt. % of an amylase preparation,
b) 5 to 50 wt. % of a protease,
c) 10 to 50 wt. % of 1,2-propylene glycol,
d) 2 to 30 wt. % of a nonionic surfactant of the general formula
R¹—CH(OH)CH₂O-(AO)_{w}-(A′O)ₓ-(A″O)_{y}-(A″O)_{z}—R²
wherein R¹ is a straight chain or branched, saturated or mono- or polyunsaturated C₆₋₂₄ alkyl or alkenyl group; R² is a linear or branched hydrocarbon group containing 2 to 26 carbon atoms; A, A′, A″ and A′″ independently are —CH₂CH₂, —CH₂CH₂—CH₂, —CH₂—CH(CH₃), —CH₂—CH₂—CH₂—CH₂, —CH₂—CH(CH₃)—CH₂—, or —CH₂—CH(CH₂—CH₃); and w, x, y and z independently are values from 0.5 to 120, wherein x, y and/or z can also be 0,
all weight percentages based on total weight of the washing or cleaning agent preparation.

10. Process for washing dishes in an automatic dishwasher comprising using a liquid washing or cleaning agent preparation according to claim 1.

11. Process according to claim 10 further comprising metering the liquid washing or cleaning agent preparation into an interior of the automatic dishwasher from a storage reservoir located in the automatic dishwasher and containing multiple amounts of the washing or cleaning agent preparation needed for carrying out a cleaning process.

12. Process according to claim 11 further comprising keeping the liquid washing or cleaning agent preparation the storage reservoir for at least two separate cleaning processes prior to being metered into the interior of the automatic dishwasher.

13. Process according to claim 11 further comprising heating the liquid washing or cleaning agent preparation in the storage reservoir at least two times to temperatures of 30° C. or greater.

14. Method of stabilizing a liquid enzyme preparation comprising:
adding a non-ionic surfactant of the general formula
R¹—CH(OH)CH₂O-(AO)_{w}-(A′O)ₓ-(A″O)_{y}-(A′″)_{z}—R²,
wherein R¹ is a straight chain or branched, saturated or mono- or polyunsaturated C₆₋₂₄ alkyl or alkenyl group; R² is a linear or branched hydrocarbon group containing 2 to 26 carbon atoms; A, A′, A″ and A′″ independently are —CH₂CH₂, —CH₂CH₂—CH₂, —CH₂—CH(CH₃), —CH₂—CH₂—CH₂—CH₂, —CH₂—CH(CH₃)—CH₂—, or —CH₂—CH(CH₂—CH₃); and w, x, y and z independently are values from 0.5 to 120, wherein x, y and/or z can also be 0,
to the liquid enzyme preparation.
